(19) United States
(12) Patent Application Publication
(10) Pub. No.: US 2024/0303780 A1
(43) Pub. Date: Sep. 12, 2024

(54) DEEP LEARNING-BASED ALGORITHM FOR REJECTING UNWANTED TEXTURES FOR X-RAY IMAGES

(71) Applicant: CANON MEDICAL SYSTEMS CORPORATION, Otawara-shi (JP)

(72) Inventors: Yi HU, Vernon Hills, IL (US); Shijie LI, Vernon Hills, IL (US); John BAUMGART, Vernon Hills, IL (US); Joseph MANAK, Vernon Hills, IL (US); Kunio SHIRAISHI, Otawara-shi Tochigi (JP); Saki HASHIMOTO, Nasushiobara Tochigi (JP)

(73) Assignee: CANON MEDICAL SYSTEMS CORPORATION, Otawara-shi (JP)

(21) Appl. No.: 18/181,635

(22) Filed: Mar. 10, 2023

(51) Int. Cl. G06T 5/00

(52) U.S. Cl. CPC G06T 5/70; G06T 5/77; G06T 2207/10016; G06T 2207/10081; G06T 2207/10121; G06T 2207/20021; G06T 2207/20081; G06T 2207/20084

(57) ABSTRACT

A medical image processing method, an X-ray diagnostic apparatus, and a method of generating a learned model includes receiving first X-ray image data, inputting the first X-ray image data to a trained model, outputting, from the trained model, an X-ray image having an image quality higher than an image quality of the first X-ray image data. The learned model was trained using contrastive learning using second X-ray image data as input data, third X-ray image data and fourth X-ray image data as label data, the third X-ray image data being negative label data having worse image quality than the second X-ray image data, and the fourth image data being positive label data having better image quality than the second X-ray image data.

### BACKGROUND

### Field

The present disclosure is directed to an image processing method, an X-ray diagnosis apparatus, and a method of generating a learned model for enhancing image quality of X-ray images. The method improves visibility of devices used in surgeries by using a deep learning algorithm that uses contrastive learning to train a network with a contrastive loss function, which includes an explicit negative loss term.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

X-ray imaging techniques utilize the transmission process of X-ray through a subject, where X-ray photons that are not absorbed by the subject reach a receptor form a shadow of the subject. The resulting static image is acquired on a receptor, which is typically a solid state detector. The X-ray images acquired can be used in two ways. (1) The raw 2D projections can be used in diagnostics (e.g. radiographs, mammography etc.) or surgical guidance (e.g. fluoroscopy, digital angiography etc.) directly. (2) The raw 2D projections taken at different angles can be used to reconstruction a 3D volume of the objects (e.g. computed tomography, cone-beam computed tomography, tomosynthesis etc.).

The image quality of X-ray images is limited by the relatively smaller number of photons reaching the receptor during the relatively short exposure time. The resolution of X-ray images is limited by the blurriness from scintillator, focal spot and geometry, Subsequently, the image quality of fluoroscopy sequences suffers from noise and blurriness problems.

Deep learning has been successfully applied to image quality improvement tasks. Despite the success of deep learning algorithms, they are difficult to control and tend to fall into unwanted solutions, and in particular, they have a hard time in learning good image quality and specifically excluding unwanted image features. Thus, there is a need for a controllable deep-learning algorithm to specifically exclude unwanted image features.

### SUMMARY

In one embodiment, there is provided an X-ray image processing method, comprising receiving first X-ray image data; inputting the first X-ray image data to a trained model; and outputting, from the trained model, an X-ray image having an image quality higher than an image quality of the first X-ray image data, wherein the trained model was trained using contrastive learning using second X-ray image data as input data, third X-ray image data and fourth X-ray image data as label data, the third X-ray image data being negative label data having worse image quality than the fourth X-ray image data, and the fourth image data being positive label data having better image quality than the second X-ray image data.

In another embodiment, there is provided an X-ray medical diagnosis apparatus, comprising processing circuitry configured to receive first X-ray image data; input the first X-ray image data to a trained model; and output, from the trained model, an X-ray image having an image quality higher than an image quality of the first X-ray image data, wherein the trained model was trained using contrastive learning using second X-ray image data as an input, and third X-ray image data and fourth X-ray image data as label data, the third X-ray image data being negative label data having worse image quality than the fourth X-ray image data, and the fourth image data being positive label data having better image quality than the second X-ray image data.

In yet another embodiment, there is provided a method of generating a trained model, comprising receiving first X-ray image data; receiving second X-ray image, the second X-ray image data being unwanted negative image data having worse image quality than the first X-ray image data; receiving third X-ray image, the third X-ray image data being wanted positive image data having better image quality than the first X-ray image data; and training the neural network model using contrastive learning using the first X-ray image data as input data and the second and third X-ray data as label data, wherein the contrastive learning includes a negative loss term for the neural network model to learn from the unwanted negative image data and a positive loss term for the neural network model to learn from the wanted positive image data.

### BRIEF DESCRIPTION OF THE DRAWINGS

FIG. 1 is a block diagram illustrating the exemplary configuration of an X-ray diagnosis apparatus;

FIG. 2 is a schematic of an implementation of a computed tomography (CT) scanner;

FIGS. 3A and 3B are flow diagrams for a data preparation pipeline, in accordance with an exemplary aspect of the disclosure;

FIGS. 4A, 4B, and 4C are flow diagrams of applying neural networks to 2D X-ray images and 3D X-ray images, in accordance with an exemplary aspect of the disclosure;

FIG. 5 is a flow diagram for a method of training a neural network by a contrastive loss that includes a positive loss and a negative loss, in accordance with an exemplary aspect of the disclosure;

FIG. 6 is a flow diagram for a method of training a neural network by a contrastive loss that includes a fixed pretrained encoder, in accordance with an exemplary aspect of the disclosure;

FIG. 7 is a flow diagram for a method of training a neural network by a contrastive loss that includes a discriminator that is the inverse of the contrastive loss, in accordance with an exemplary aspect of the disclosure;

FIG. 8 is a flow diagram for deep learning-based image restoration, in accordance with an exemplary aspect of the disclosure;

FIG. 9 is a flow diagram for deep learning-based image restoration that includes pruning and precision reduction for real-time inferencing, in accordance with an exemplary aspect of the disclosure;

FIG. 10 is a flow diagram for deep learning-based image restoration that is implemented on multiple processors for real-time inferencing, in accordance with an exemplary aspect of the disclosure; and

FIG. 11 is a block diagram illustrating an example computer system for implementing the machine learning training and inference methods according to an exemplary aspect of the disclosure.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

The image quality of 2D X-ray images (e.g., fluoroscopy, X-ray images, cone-beam CT/CT projection etc.) and reconstructed 3D images (e.g., cone-beam CT or CT volumes, etc.) suffers from noise and blurriness problems. Projection according to the present disclosure relates to a scan that produces a 2D image. Reconstruction according to the present disclosure relates to creation of a 3D image from several scans from different angles.

As an example of noise and blurriness, devices used in interventional surgeries (e.g., stents, guidewires, etc.) can be made visible using X-ray fluoroscopic images. However, image quality of fluoroscopic sequences suffers from noise and blurriness. Deep learning networks have improved in their capability of image classification, as well as have been shown to be effective in reducing noise and blurriness, but still have a tendency to fall into unwanted solutions during training. For example, an image that has poor image quality can be mistakenly considered as being of good quality during training. In particular, it is difficult to control a deep learning algorithm to improve image quality. The disclosure relates to controllable deep learning to specifically exclude unwanted image appearance. In one embodiment, the deep learning excludes unwanted image appearance by using contrastive learning. The disclosure also provides a data preparation pipeline to collect or simulate unwanted images.

Radiation according to the present disclosure can include not only a-rays, ß-rays, and y-rays that are beams generated by particles (including photons) emitted by radioactive decay, but also beams having equal or more energy, for example, X-rays, particle rays, and cosmic rays.

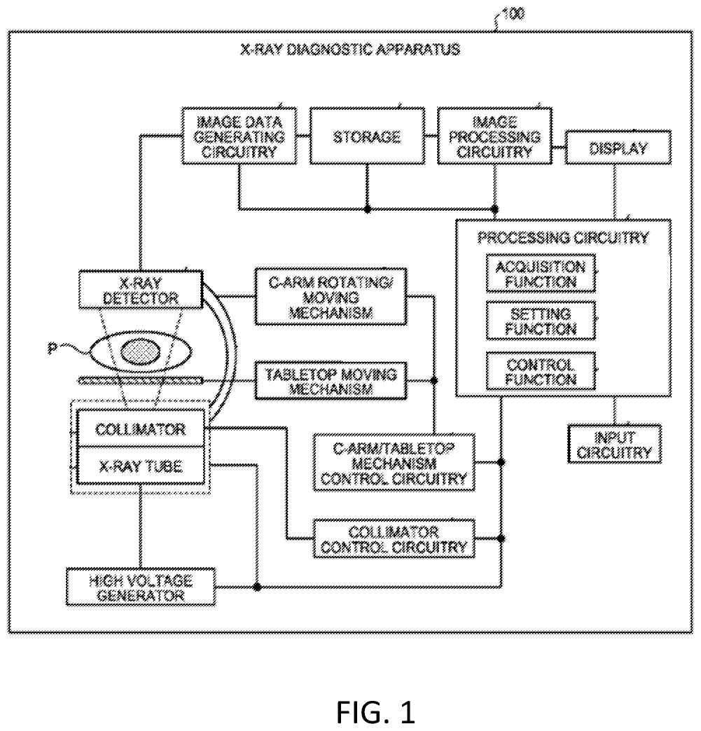

In one embodiment, an X-ray diagnostic apparatus can be an X-ray diagnostic apparatus with a C-arm. FIG. 1 is a block diagram illustrating an exemplary configuration of the X-ray diagnosis apparatus 100.

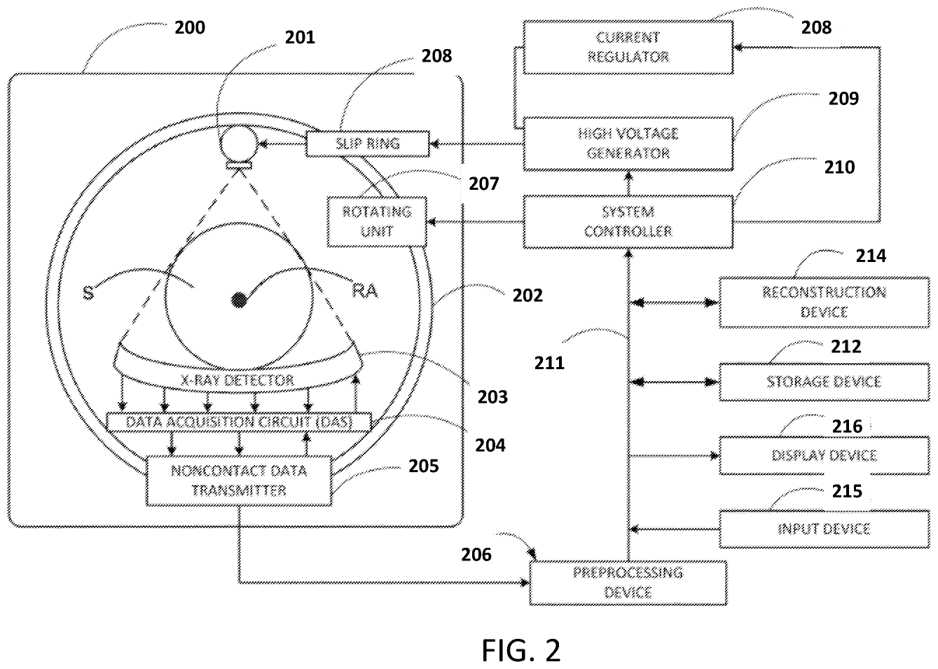

FIG. 2 is a schematic of an implementation of a computed tomography (CT) scanner. As shown in FIG. 2, a radiography gantry 200 is illustrated from a side view and further includes an X-ray tube 201, an annular frame 202, and a multi-row or two-dimensional-array-type X-ray detector 203. The X-ray tube 201 and X-ray detector 203 are diametrically mounted across an object OBJ on the annular frame 202, which is rotatably supported around a rotation axis RA. A rotating unit 207 rotates the annular frame 202 at a high speed, such as 0.4 see/rotation, while the object OBJ is being moved along the axis RA into or out of the illustrated page.

The embodiment of an X-ray computed tomography (CT) apparatus according to the present inventions will be described below with reference to the views of the accompanying drawing. Note that X-ray CT apparatuses include various types of apparatuses, e.g., a rotate/rotate-type apparatus in which an X-ray tube and X-ray detector rotate together around an object to be examined, and a stationary/rotate-type apparatus in which many detection elements are arrayed in the form of a ring or plane, and only an X-ray tube rotates around an object to be examined. The present inventions can be applied to either type. In this case, the rotate/rotate type, which is currently the mainstream, will be exemplified.

The multi-slice X-ray CT apparatus further includes a high voltage generator 209 that generates a tube voltage applied to the X-ray tube 201 through a slip ring 208 so that the X-ray tube 201 generates X-rays. The X-rays are emitted towards the object OBJ, whose cross-sectional area is represented by a circle. For example, the X-ray tube 201 having an average X-ray energy during a first scan that is less than an average X-ray energy during a second scan. Thus, two or more scans can be obtained corresponding to different X-ray energies. The X-ray detector 203 is located at an opposite side from the X-ray tube 201 across the object OBJ for detecting the emitted X-rays that have transmitted through the object OBJ. The X-ray detector 203 further includes individual detector elements or units.

The CT apparatus further includes other devices for processing the detected signals from X-ray detector 203. A data acquisition circuit or a Data Acquisition System (DAS) 204 converts a signal output from the X-ray detector 203 for each channel into a voltage signal, amplifies the signal, and further converts the signal into a digital signal. The X-ray detector 203 and the DAS 204 are configured to handle a predetermined total number of projections per rotation (TPPR).

The above-described data is sent to a preprocessing device 206, which is housed in a console outside the radiography gantry 200 through a non-contact data transmitter 205. The preprocessing device 206 performs certain corrections, such as sensitivity correction on the raw data. A memory 212 stores the resultant data, which is also called projection data at a stage immediately before reconstruction processing. The memory 212 is connected to a system controller 210 through a data/control bus 211, together with a reconstruction device 214, input device 215, and display 216. The system controller 210 controls a current regulator 213 that limits the current to a level sufficient for driving the CT system.

The detectors are rotated and/or fixed with respect to the patient among various generations of the CT scanner systems. In one implementation, the above-described CT system can be an example of a combined third-generation geometry and fourth-generation geometry system. In the third-generation system, the X-ray tube 201 and the X-ray detector 203 are diametrically mounted on the annular frame 202 and are rotated around the object OBJ as the annular frame 202 is rotated about the rotation axis RA. In the fourth-generation geometry system, the detectors are fixedly placed around the patient and an X-ray tube rotates around the patient. In an alternative embodiment, the radiography gantry 200 has multiple detectors arranged on the annular frame 202, which is supported by a C-arm and a stand.

The memory 212 can store the measurement value representative of the irradiance of the X-rays at the X-ray detector unit 203. Further, the memory 212 can store a dedicated program for executing various steps of method 100 and/or method 100′ for correcting low-count data and CT image reconstruction.

The reconstruction device 214 can execute various steps of method 100 and/or method 100′. Further, reconstruction device 214 can execute pre-reconstruction processing image processing such as volume rendering processing and image difference processing as needed.

The pre-reconstruction processing of the projection data performed by the preprocessing device 206 can include correcting for detector calibrations, detector nonlinearities, and polar effects, for example. Further, the pre-reconstruction processing can include various steps of method 100 and/or method 100′.

Post-reconstruction processing performed by the reconstruction device 214 can include filtering and smoothing the image, volume rendering processing, and image difference processing as needed. The image reconstruction process can implement various of the steps of method 100 and/or method 100′ in addition to various CT image reconstruction methods. The reconstruction device 214 can use the memory to store, e.g., projection data, reconstructed images, calibration data and parameters, and computer programs.

The reconstruction device 214 can include a CPU (processing circuitry) that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory 212 can be non-volatile, such as ROM, EPROM, EEPROM or FLASH memory. The memory 212 can also be volatile, such as static or dynamic RAM, and a processor, such as a microcontroller or microprocessor, can be provided to manage the electronic memory as well as the interaction between the FPGA or CPLD and the memory.

Alternatively, the CPU in the reconstruction device 214 can execute a computer program including a set of computer-readable instructions that perform the functions described herein, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xenon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

In one implementation, the reconstructed images can be displayed on a display 216. The display 216 can be an LCD display, CRT display, plasma display, OLED, LED or any other display known in the art.

The memory 212 can be a hard disk drive, CD-ROM drive, DVD drive, FLASH drive, RAM, ROM or any other electronic storage known in the art.

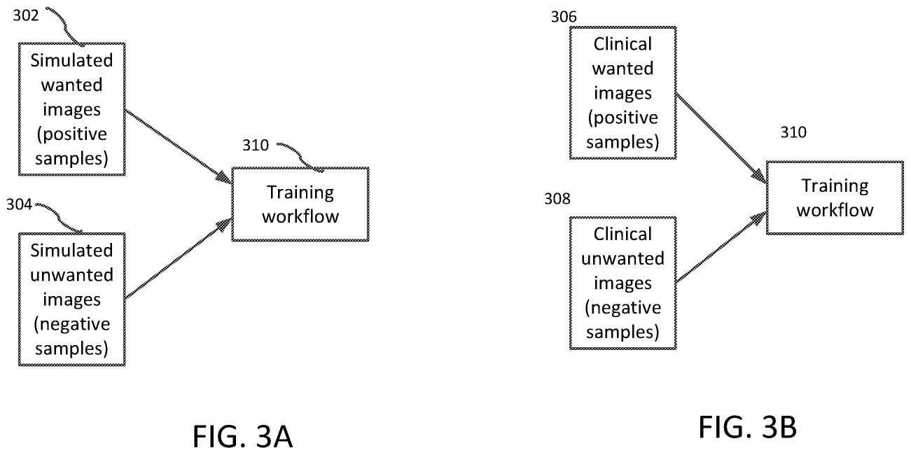

FIGS. 3A and 3B are flow diagrams for a data preparation pipeline, in accordance with an exemplary aspect of the present disclosure. Medical images are often blurry and noisy making them difficult to interpret. For example, fluoroscopic images tend to show blurry edges and overly unclear textures, due in part to noise. Conventional positive-loss-based deep-learning image restoration algorithms tend to over-smooth images, due in part to accepting unwanted images as being positive. Medical images, such as fluoroscopic images, of high image quality are those that have clear edges and accurate texture.

In a data preparation stage, unwanted images (negative samples) are prepared as well as positive images (positive samples). In development of disclosed embodiments, it has been determined that increasing the number of, and explicit rejection of, unwanted images results in medical images that are more accurately smoothed and have clearer edges. Unwanted images 308 can be selected from actual clinical images, but unwanted images 304 can also be obtained through simulation.

For purposes of this disclosure, unwanted images (negative samples) are images that have general blurriness in at least one of edges and texture, and/or are images with at least one artifact. Unwanted images 304 can be obtained through simulation by simulating a blurred image from a good quality image, adding arbitrary artifacts to the good quality image, as well as adding noise to reduce image quality. Blurriness is simulated by adjusting texture and softening edges. Artifacts can be extracted from clinical images and incorporated into simulated images. Artifacts can also be generated by the simulation based on other known artifacts or may be manually produced and incorporated into the simulation.

For purposes of this disclosure, a wanted image (positive sample) is one that is substantially free of artifacts and blurriness such that edges and texture are visually clear and accurate. Wanted images 302 can also be obtained through simulation, as well as selected from actual clinical images 306. One approach to obtaining wanted images is to start with a good quality image and simulate movement of the good quality image. Another approach to obtaining wanted images is to generate different views of a good quality image. In this disclosure, image quality can be used to distinguish wanted images from unwanted images. Positive samples are wanted images having better image quality than unwanted images (negative samples) in terms of quantitative measurements (e.g., less noise, less blurriness, higher resolution, artifact-free, etc.). However, other criteria can be used to determine wanted and unwanted images used for training.

A deep learning network 310 is trained using combinations of simulated and clinical images, simulated images only, or clinical images only, depending on the availability of positive and unwanted images. In one embodiment, the deep learning network 310 is trained with at least one unwanted image. It is preferred that each unwanted (negative) image in a training set have at least one corresponding positive image.

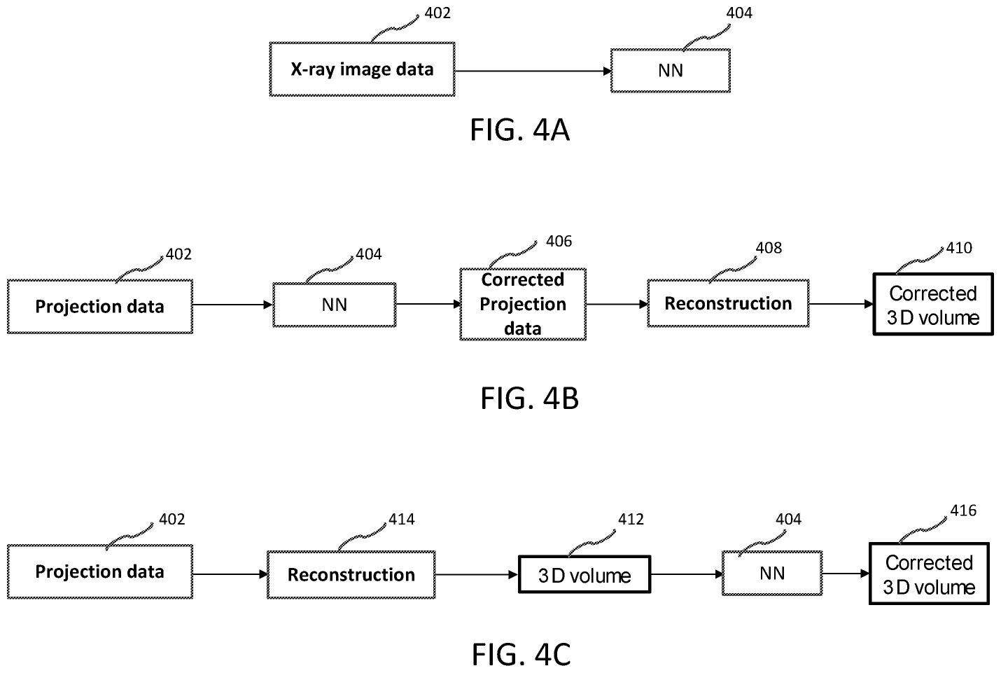

FIGS. 4A, 4B, and 4C are flow diagrams of applying neural networks to 2D medical images and 3D medical images, in accordance with an exemplary aspect of the disclosure. In FIG. 4A, in the case of 2D medical images, the neural network 404 is applied directly to the projection data 402. For purposes of this disclosure, X-ray images are reconstructed from a number of projections that are acquired as an X-ray tube rotates through 360° around the object (patient).

In FIG. 4B, for 3D medical images, the neural network 404 is applied to the projection data 402 to obtain corrected projection data 406, before reconstruction 408, to obtain a corrected 3D volume 410.

In FIG. 4C, in another embodiment for 3D medical images, the neural network 404 is applied to the reconstruction 3D volume 412 after reconstruction 414, to obtain corrected 3D volume 416. For purposes of this disclosure, cone-beam computed tomography (CBCT) is a radiographic imaging method for obtaining three-dimensional (3D) imaging of hard tissue structures.

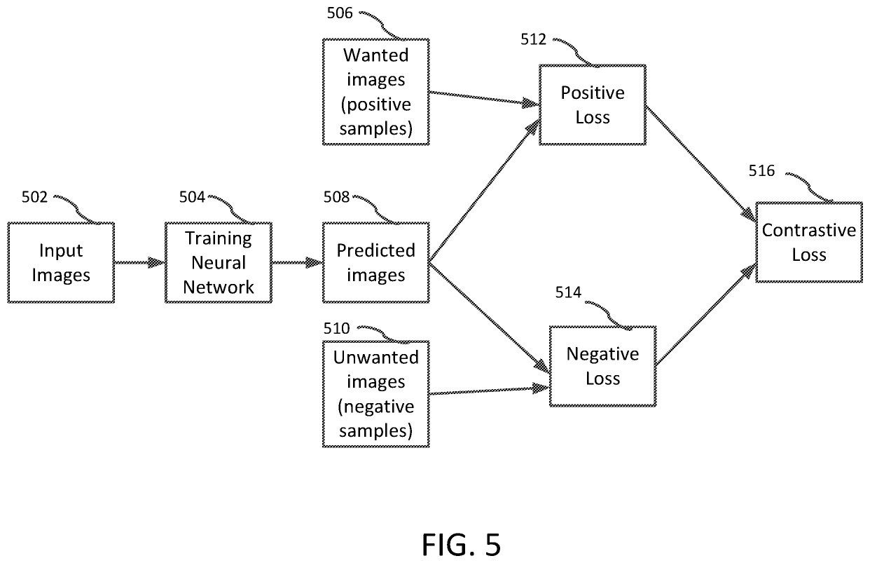

FIG. 5 is a flow diagram for a method of training a neural network by contrastive loss that includes a positive loss and a negative loss, in accordance with an exemplary aspect of the present disclosure. All images of a training set 502, including wanted images (positive samples) and unwanted images (negative samples) with poor image quality, are input for training a deep learning network 504. Images can be input for training one image at a time, or as a batch (or mini-batch) of training data.

The neural network 504 can be any neural network configured for image restoration, where the input is an image and the output is an image having been generated by the neural network as a predicted image 508 with improved image quality. A set of wanted images (positive samples) 506 and a set of unwanted images (negative samples) 510 are used in the calculation of a contrastive loss 516. The contrastive loss is fed back to update the neural network 504 during training. The neural network 504 is trained by contrastive learning in which input images 502 are input as pairs of a positive sample and a negative sample. In one embodiment, the input images 502 are input as a positive sample and a corresponding set of multiple negative samples. The multiple negative samples represent unwanted images for an image that is a positive sample.

Referring to FIG. 5, the contrastive loss term 516 includes a negative loss term 514 based on the predicted output 508 of the neural network 504 and unwanted images 510, as well as a positive loss term 512 based on a difference between the predicted output 508 and wanted images 506. The contrastive loss term can also include a term that is the difference between the input image 502 and the predicted image 508. A difference function d( ) can be a Mean Absolute Error (MAE) or a Mean Squared Error (MSE), which is also referred to as Root Mean-Squared Error. These errors represent the differences between the predicted values (values predicted by the neural network) and the actual values. In one embodiment, the contrastive loss is determined as:

$$Contrastive Loss = \frac{d ( Y , P )}{d ( Y , N )} + \frac{d ( Y , P )}{d ( Y , X )}$$

where d( ) is MAE or MSE, Y is the output of the neural network, P refers to the positive samples, N refers to the negative samples, and X is the input image.

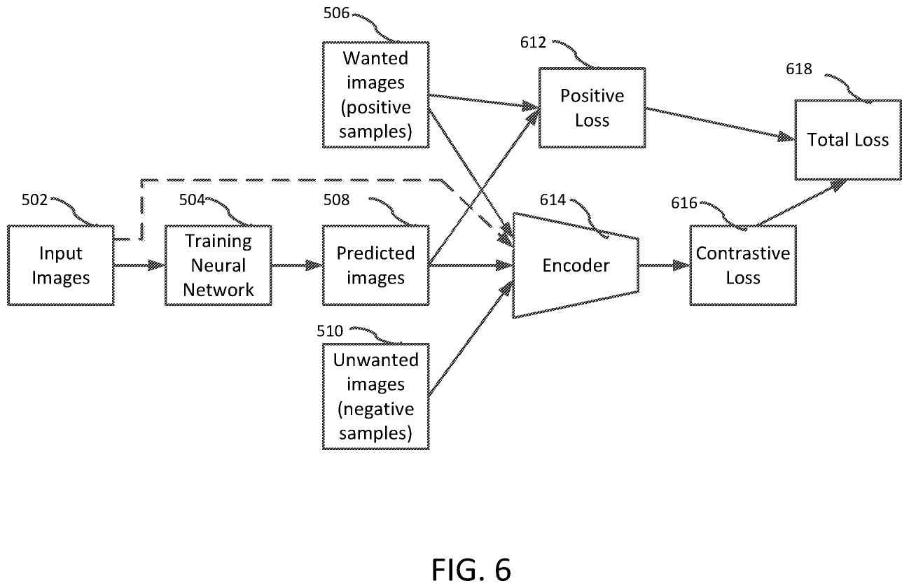

FIG. 6 is a flow diagram for a method of training a neural network by a contrastive loss method that includes a fixed pretrained encoder, in accordance with an exemplary aspect of the present disclosure. The encoder may be a fully connected network with a number of layers L. In FIG. 6, the total loss function 618 includes a fixed pretrained encoder 614 to encode a wanted image 506, a predicted image 508, and an unwanted image 510 into respective encoded images. A positive loss 612 is added to the contrastive loss to generate a total loss. The contrastive loss can be computed as:

$$Contrastive Loss = {\sum }_{l = 1}^{L} {w}_{l} \frac{d ( {E ( Y )}^{l} , {E ( P )}^{l} )}{d ( {E ( Y )}^{l} , {E ( N )}^{l} )}$$

where E is a fixed pretrained encoder, d( ) is MAE or MSE, Y is the output of the neural network, P refers to the positive samples, N refers to the negative samples, X is the input image, L refers to the number of intermediate layers in the encoder, and w₁ is a weight for each intermediate layer.

In one embodiment, specific features in one or more unwanted images can be emphasized using a mask.

In one embodiment, the encoder 614 is configured with an additional projection layer and a normalization layer. The projection layer can receive inputs from a wanted image 506, a predicted image 508, and an unwanted image 510, which are mapped to a vector space of a reduced dimension. A projection layer is typically a small neural network, e.g., an MLP with one hidden layer, that is used to map the representations from the base encoder to a reduced dimensional latent space.

The normalization layer normalizes the input across the features. Normalization is used for training the neural network so that the different features are on a similar scale.

In one embodiment, the difference is determined as an exponentiation of a first term “a” multiplied by a second term “b”, divided by a constant “tau”.

The contrastive loss 616 is determined using results from the encoder 614. The positive loss 612 is determined using a difference between the predicted image 508 and the wanted image 506. In another embodiment, the contrastive loss can be computed as:

$$Contrastive Loss = {\sum }_{l = 1}^{L} {w}_{l} \frac{d ( {E ( Y )}^{l} , {E ( P )}^{l} )}{d ( {E ( Y )}^{l} , {E ( N )}^{l} ) + d ( {E ( Y )}^{l} , {E ( P )}^{l} )} \\ d ( a , b ) := exp ( a \cdot b / τ )$$

where E is a fixed pretrained encoder with an additional projection layer and normalization layer, d(a,b) is a dot product similarity function where t is a constant, referred to as a temperature scalar, Y is the output of the neural network, P refers to the positive samples, N refers to the negative samples, X is the input image, L refers to the number of intermediate layers in the encoder, and w₁ is a weight for each intermediate layer.

In one embodiment, a contrastive loss method includes a fixed pretrained encoder and a logarithmic function. In the one embodiment, the encoder 614 of the total loss 618 applies a weighted logarithm to each intermediate layer of an encoder 614 of L layers. The logarithmic function helps to keep the weighted values low. The contrastive loss 616 is determined using results from the encoder 614. The positive loss 612 is determined using a difference between the predicted image 508 and the wanted image 506. In one embodiment, he contrastive loss is computed as:

$$Contrastive Loss = - {\sum }_{l = 1}^{L} {w}_{l} log ( \frac{d ( {E ( Y )}^{l} , {E ( P )}^{l} )}{d ( {E ( Y )}^{l} , {E ( N )}^{l} )} + \frac{d ( {E ( Y )}^{l} , {E ( P )}^{l} )}{d ( {E ( Y )}^{l} , {E ( X )}^{l} )} )$$

where E is a fixed pretrained encoder, d( ) is MAE or MSE, Y is the output of the neural network, P refers to the positive samples, N refers to the negative samples, X is the input image, L refers to the number of intermediate layers in the encoder, and w₁ is a weight for each intermediate layer.

In one embodiment, a contrastive loss method includes a fixed pretrained encoder with an additional projection and normalization and a logarithmic function. The total loss 618 includes an encoder 614. The encoder 614 is configured with an additional projection layer and a normalization layer. The total loss 618 includes a difference that is determined based on an exponential of a term “a” multiplied by a term “b”, over a constant “tau.” The contrastive loss 616 is determined using results from the encoder 614. The positive loss 612 is determined using a difference between the predicted image 508 and the wanted image 506. In one embodiment, the contrastive loss is computed as:

$$Contrastive Loss = - {\sum }_{l = 1}^{L} {w}_{l} log \frac{d ( {E ( Y )}^{l} , {E ( P )}^{l} )}{d ( {E ( Y )}^{l} , {E ( N )}^{l} ) + d ( {E ( Y )}^{l} , {E ( P )}^{l} ) + d ( {E ( Y )}^{l} , {E ( X )}^{l} )} \\ d ( a , b ) := exp ( a \cdot b / τ )$$

where E is a fixed pretrained encoder with an additional projection layer and normalization layer, d(a,b) is a dot product similarity function where t is a constant, referred to as a temperature scalar, Y is the output of the neural network, P refers to the positive samples, N refers to the negative samples, X is the input image, L refers to the number of intermediate layers in the encoder, and w₁ is a weight for each intermediate layer.

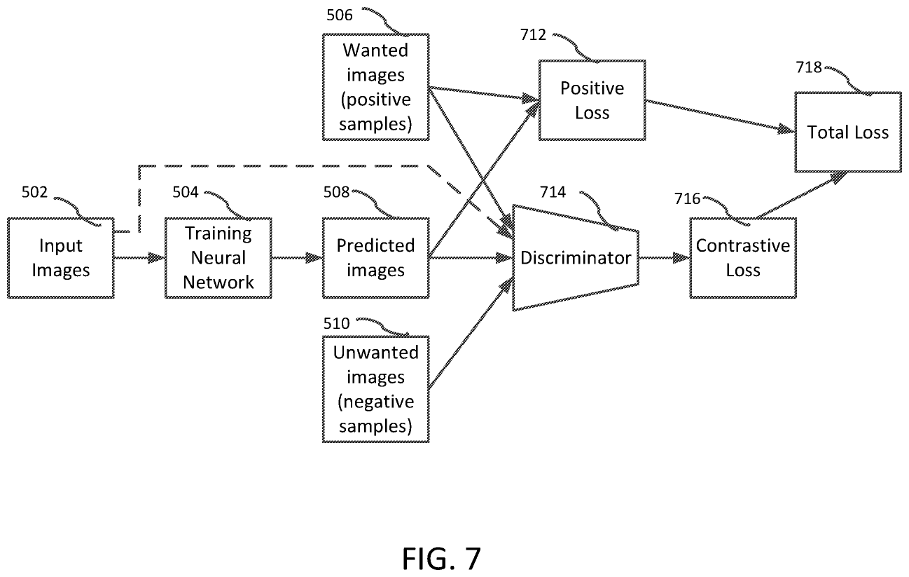

FIG. 7 is a flow diagram for a method of training a neural network by a contrastive loss method that includes a discriminator that is the inverse of the contrastive loss, in accordance with an exemplary aspect of the disclosure. In one embodiment, the contrastive loss 716 is determined based on a trainable discriminator 714. The discriminator 714 is a neural network that is trained based on a loss that is the inverse of the contrastive loss 716. The neural network 504 is trained based on a sum of the positive loss and the contrastive loss. The discriminator 714 is trained together with NN 504. In one iteration, discriminator 714 is fixed and then NN 504 is tunable, and then in the next iteration, NN 504 is fixed and then discriminator 714 is under training. That being said, discriminator 714 and NN 504 are trained alternatively. As the discriminator neural network 714 is trained, it enhances differences in negative samples from among the predicted image, the unwanted images, and the wanted image. The contribution to negative loss will be larger for negative samples.

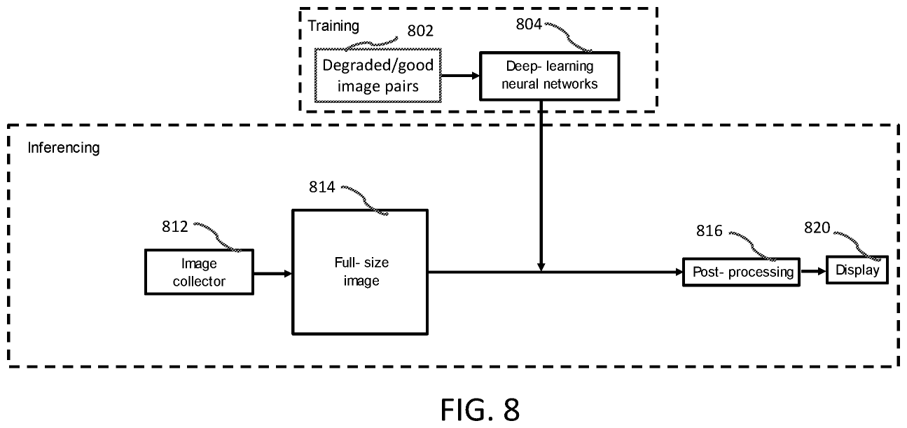

FIG. 8 is a flow diagram for deep-learning based image restoration, in accordance with an exemplary aspect of the disclosure. In order to perform inferencing, the trained neural network is used to output a predicted image based on an input image 814. The input image 814 can be a full-size image obtained from a storage device, as an image collector 812. The predicted image can be post processed in a post processor 816 in order to be displayed on a display device 820. The neural network is trained on unwanted and positive image pairs 802 using an embodiment of the deep learning method 804.

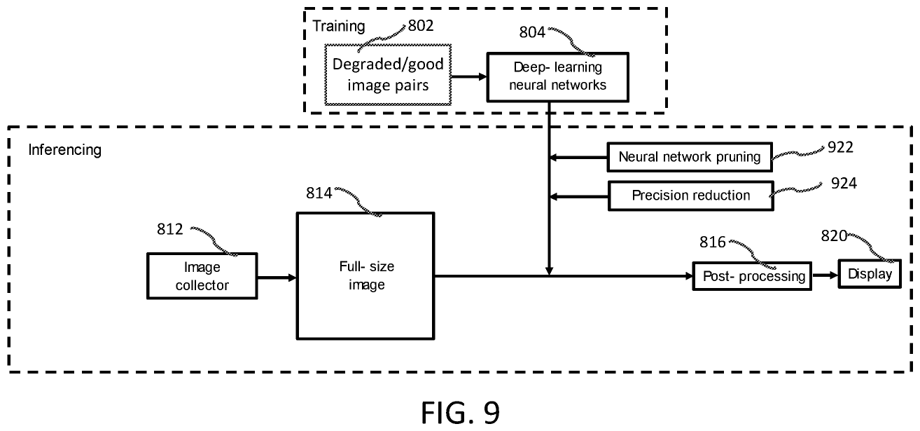

FIG. 9 is a flow diagram for deep-learning based image restoration that includes pruning and precision reduction for real-time inferencing, in accordance with an exemplary aspect of the disclosure. In order to speed up inferencing, the trained neural network may be pruned by a neural network pruning component 922 to remove unnecessary weighted connections, such as weighted connections that are below a predetermined value. The trained neural network may be further subject to precision reduction by a precision reduction component 924, either due to limitations of the processor, or to improve processing speed through simpler multiply-add computations. Precision reduction can be made by limiting the number of decimal places in weighted connections.

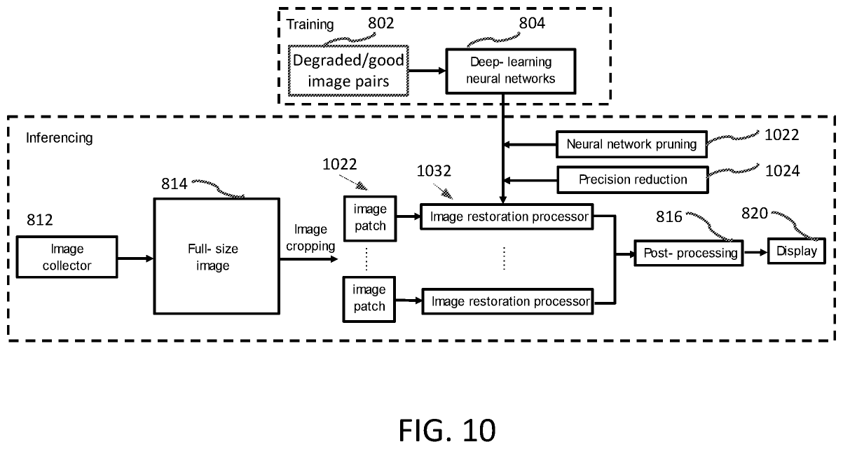

FIG. 10 is a flow diagram for deep-learning based image restoration that is implemented on multiple processors for real-time inferencing, in accordance with an exemplary aspect of the disclosure. In order to further speed up inferencing and make use of a processor with multiple cores, the input image 814 may be divided into multiple image patches 1022 by an image pre-processor, and the image patches are fed to different image restoration processors 1032 for inferencing to generate restored image patches. The different image restoration processors 1032 can be configured based on the deep learning network 804 that has been trained using an embodiment of the deep learning method. The deep learning network 804 can be pruned by a pruning component 1022 and be subject to precision reduction by a precision reduction component 1024. The different image processors can be configured to run in parallel. The image post-processor 816 can piece together the restored image patches to output a restored full image to the display device 820.

The disclosed embodiments of deep learning produce sharper images with clearer edges and more accurate texture without over-smoothing. The contrastive learning of the disclosed embodiments enables improved control of image quality compared to simply relying on good image quality as a learning criteria. The contrastive loss function explicitly discourages production of output data similar to the negative training data. To ensure better correlation between positive training data and negative training data, unwanted images can either be collected from clinical settings or can be obtained using simulation.

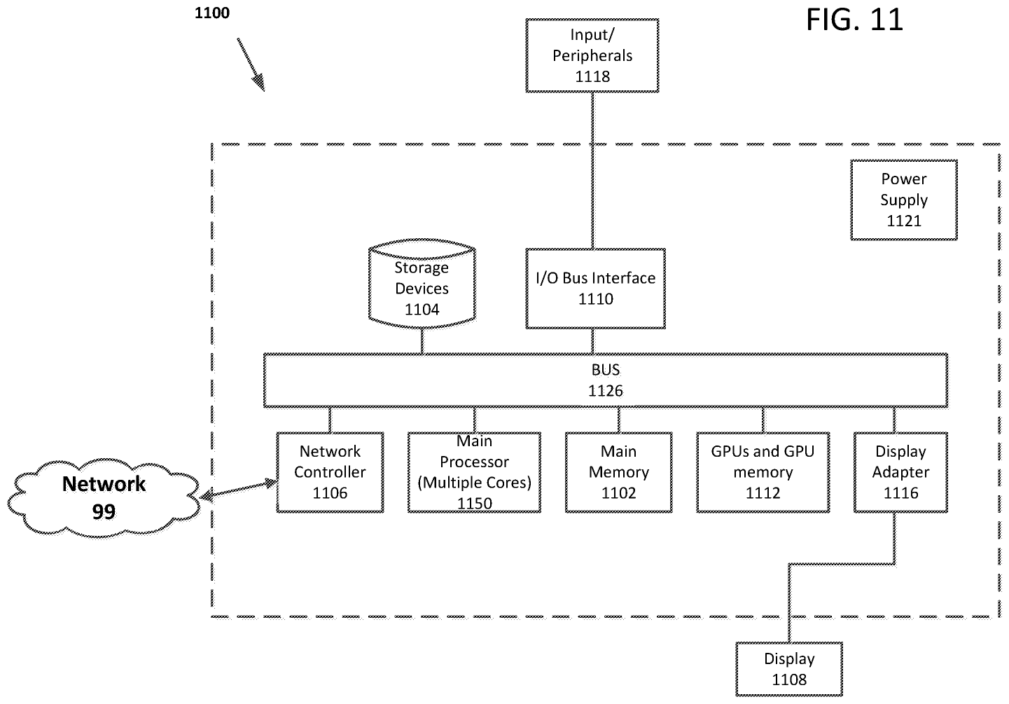

FIG. 11 is a block diagram illustrating an example computer system for implementing the machine learning training and inference methods according to an exemplary aspect of the disclosure. In a non-limiting example, the computer system can be an AI workstation running an operating system, for example Ubuntu Linux OS, Windows, a version of Unix OS, or Mac OS. The computer system 1100 can include one or more central processing units (CPU) 1150 having multiple cores. The computer system 1100 can include a graphics board 1112 having multiple GPUs, each GPU having GPU memory. The graphics board 1112 can perform many of the mathematical operations of the disclosed machine learning methods. The computer system 1100 includes main memory 1102, typically random access memory RAM, which contains the software being executed by the processing cores 1150 and GPUs 1112, as well as a non-volatile storage device 1104 for storing data and the software programs. Several interfaces for interacting with the computer system 1100 may be provided, including an I/O Bus Interface 1110, Input/Peripherals 1118 such as a keyboard, touch pad, mouse, Display Adapter 1116 and one or more Displays 1108, and a Network Controller 1106 to enable wired or wireless communication through a network 99. The interfaces, memory and processors may communicate over the system bus 1126. The computer system 1100 includes a power supply 1121, which may be a redundant power supply.

In one embodiment, the computer system 1100 includes a mult-core CPU and a graphics card by NVIDIA, in which the GPUs have multiple cores. In one embodiment, the computer system 1100 may include a machine learning engine 1112.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. An X-ray image processing method, comprising:
receiving first X-ray image data;
inputting the first X-ray image data to a trained model; and
outputting, from the trained model, an X-ray image having an image quality higher than an image quality of the first X-ray image data,
wherein the trained model was trained using contrastive learning using second X-ray image data as input data, third X-ray image data and fourth X-ray image data as label data, the third X-ray image data being negative label data having worse image quality than the fourth X-ray image data, and the fourth image data being positive label data having better image quality than the second X-ray image data.

2. The method of claim 1, wherein the third X-ray image data includes X-ray image data with blurriness.

3. The method of claim 1, wherein the positive label data used in the training is X-ray image data with less noise and blurriness than the second X-ray image data.

4. The method of claim 1, wherein the second X-ray image data is image data with noise and blurriness, and the fourth X-ray image data is image data with less noise and less blurriness than the second X-ray image data.

5. The method of claim 1, wherein the contrastive learning uses a negative loss function term to learn from unwanted negative images used for the third X-ray image data.

6. The method of claim 1, wherein the contrastive learning simultaneously uses a negative loss term and a positive loss term.

7. The method of claim 5, wherein the contrastive learning includes encoding positive images, images predicted by the trained model, and the unwanted negative images so as to increase a weight for specific features.

8. The method of claim 7, wherein the encoding includes passing the images through a projection layer.

9. The method of claim 1, wherein the contrastive learning includes training a discriminator on an inverse of the contrastive loss using, as input to the discriminator, positive images, images predicted by the trained model, and the negative label data.

10. An X-ray medical diagnosis apparatus, comprising:
processing circuitry configured to
receive first X-ray image data;
input the first X-ray image data to a trained model; and
output, from the trained model, an X-ray image having an image quality higher than an image quality of the first X-ray image data,
wherein the trained model was trained using contrastive learning using second X-ray image data as an input, and third X-ray image data and fourth X-ray image data as label data, the third X-ray image data being negative label data having worse image quality than the fourth X-ray image data, and the fourth image data being positive label data having better image quality than the second X-ray image data.

11. The X-ray medical diagnosis apparatus of claim 10, wherein the processing circuitry is further configured to receive, as the first X-ray image data, X-ray fluoroscopy image data from a sequence of fluoroscopy images obtained by an image collector.

12. The X-ray medical diagnosis apparatus of claim 10, wherein the processing circuitry is further configured to:
remove, from the trained neural network, weighted connections that are below a predetermined value, and
reduce a precision of the weighted connections of the trained neural network.

13. The X-ray medical diagnosis apparatus of claim 10, wherein the processing circuitry includes multiple processors and an image preprocessor;
the image preprocessor is configured to divide the first X-ray image data into a plurality of patches of image data, and
the multiple processors are configured to, based on the trained model, receive a subset of the plurality of patches of image data and generate respective restored patches of image data.

14. A method of generating a trained model, comprising:
receiving first X-ray image data;
receiving second X-ray image, the second X-ray image data being unwanted negative image data having worse image quality than the first X-ray image data;
receiving third X-ray image, the third X-ray image data being wanted positive image data having better image quality than the first X-ray image data; and
training the neural network model using contrastive learning using the first X-ray image data as input data and the second and third X-ray data as label data,
wherein the contrastive learning includes a negative loss term for the neural network model to learn from the unwanted negative image data and a positive loss term for the neural network model to learn from the wanted positive image data.

15. The method of claim 14, wherein the contrastive learning simultaneously uses the negative loss term in combination with the positive loss term.

16. The method of claim 14, wherein the contrastive learning includes encoding positive images, images predicted by the trained model, and the unwanted negative image data so as to increase a weight for specific features.

17. The method of claim 16, wherein the encoding includes passing the predicted images through a projection layer.

18. The method of claim 14, wherein the contrastive learning includes training a discriminator on an inverse of the contrastive loss using, as input to the discriminator, positive image data, an image predicted by the trained model, and the unwanted negative image data.
